(19) United States
(12) Patent Application Publication
(10) Pub. No.: US 2007/0249623 A1
(43) Pub. Date: Oct. 25, 2007

(54) 1-(Indole-6-Carbonyl-D-Phenylglycinyl)-4-(1-Methylpiperidin-4-Yl)Piperazine D-Tartrate

(75) Inventor: Julie Kay Bush, Fishers, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company Patent Division, Indianapolis, IN (US)

(21) Appl. No.: 11/570,686

(22) PCT Filed: Jun. 13, 2005
(86) PCT No.: PCT/US05/20490

§ 371 Date: Dec. 15, 2006

Related U.S. Application Data
(60) Provisional application No. 60/583,599, filed on Jun. 30, 2004.

(51) Int. Cl. A61K 31/497; A61P 7/02; C07D 401/00

(52) U.S. Cl. 514/253.01; 544/360

(57) ABSTRACT

1-(Indole-6-carbonyl-D-phenylglycinyl)-4-(1-methyl-piperidin-4-yl)piperazine D-tartrate forms a stable crystalline salt and is an inhibitor of the serine protease, Factor Xa, useful in the treatment of cardiovascular disorders.

This invention relates to a pharmaceutical compound that is a selective inhibitor of the serine protease, Factor Xa, to pharmaceutical compositions thereof and to its use in the treatment of the human or animal body.

The serine proteases are a group of proteolytic enzymes which have a common catalytic mechanism characterized by a particularly reactive Ser residue. Examples of serine proteases include trypsin, tryptase, chymotrypsin, elastase, thrombin, plasmin, kallikrein, Complement Cl, acrosomal protease, lysosomal protease, cocoonase, α-lytic protease, protease A, protease B, serine carboxypeptidase II, subtilisin, urokinase, Factor VIIa, Factor IXa, and Factor Xa. The serine proteases have been investigated extensively over a period of several decades and the therapeutic value of inhibitors of serine proteases is well understood. Thus, for example, an inhibitor of Factor Xa has value as a therapeutic agent as an anticoagulant, e.g. in the treatment and prevention of thrombotic disorders. The use of a Factor Xa inhibitor as an anticoagulant is desirable in view of the selectivity of its effect. Many clinically approved anticoagulants have been associated with adverse events owing to the non-specific nature of their effects on the coagulation cascade.

WO 00/76971 discloses that the compound 1-(indole-6-carbonyl-D-phenylglycinyl)-4-(1-methylpiperidin-4-yl)-piperazine is a potent and selective inhibitor of Factor Xa with particularly desirable biological properties. The compound and its pharmaceutically acceptable salts are therefore potentially useful for the prophylaxis or treatment of thrombotic disorders such as amongst others venous thrombosis, pulmonary embolism, arterial thrombosis, myocardial ischaemia, myocardial infarction, and cerebral thrombosis, including prevention of stroke in atrial fibrillation. They also potentially have benefit in the treatment of acute vessel closure associated with thrombolytic therapy and restenosis, e.g. after transluminal coronary angioplasty or bypass grafting of the coronary or peripheral arteries and in the maintenance of vascular access patency in long term hemodialysis patients.

In order to be considered as a candidate for further development as a pharmaceutical, a compound must not only possess desirable biological properties, but also physical properties that adapt it for use in the manufacture of a pharmaceutical product. In particular, the compound should form a stable, preferably crystalline, solid that can readily be manufactured and formulated.

It has been proved to be particularly difficult to find stable, crystalline forms of 1-(indole-6-carbonyl-D-phenyl-glycinyl)-4-(1-methylpiperidin-4-yl)piperazine.

Salts of 1-(indole-6-carbonyl-D-phenylglycinyl)-4-(1-methylpiperidin-4-yl)piperazine that form crystalline solids are disclosed in Examples 48a (hydrochloride salt) and 48b (difumarate salt) of WO 01/96323. WO 02/100847, which claims priority from WO 01/96323, notes that the hydrochloride salt has been found to have disadvantageous properties and further discloses that the difumarate salt can exist in more than one crystalline form, each of which is claimed in the application. The difumarate salt provides crystals that have superior properties to crystals of the hydrochloride salt. However, further experience of working with these has revealed the need for a salt with further improved properties. Thus, the form disclosed in WO 02/100847 as Form 1 is obtained as thin needles. The thin needle morphology has disadvantages for formulation, however, because of, inter alia, clustering and low bulk density. Further, Form 1 has been found to convert to a different (higher) hydrate under conditions of extremely high (above 80%) relative humidity and to convert into the form disclosed in WO 02/100847 as Form 2 in aqueous suspensions. The form disclosed in WO 02/100847 as Form 2 has the disadvantage, inter alia, that the particle size is very small, resulting in extremely slow filtration.

Thus, there remains a need for a salt form of 1-(indole-6-carbonyl-D-phenylglycinyl)-4-(1-methylpiperidin-4-yl)-piperazine that forms a crystalline solid that has a desirable morphology, is stable in the presence of water and under conditions of very high relative humidity (above 80%), and can readily be prepared on a large scale.

A new salt of 1-(indole-6-carbonyl-D-phenylglycinyl)-4-(1-methylpiperidin-4-yl)piperazine has now been found that can readily be prepared in a stable, crystalline form.

Thus, viewed from one aspect, the invention provides 1-(indole-6-carbonyl-D-phenylglycinyl)-4-(1-methylpiperidin-4-yl)piperazine D-tartrate, which may also be denoted as 1-(indole-6-carbonyl-D-phenylglycinyl)-4-(1-methylpiperidin-4-yl)piperazine D-tartaric acid salt (1:1).

It will be appreciated that the basic compound may exist in racemic (D/L) or chiral form, and that the preferred D-isomer may be administered in a racemic mixture with the L-isomer, or alone. The D-configuration refers to the configuration of D-phenylglycine, from which the compound may be prepared.

According to another aspect, the present invention provides 1-(indole-6-carbonyl-D-phenylglycinyl)-4-(1-methyl-piperidin-4-yl)piperazine D-tartrate in crystalline form.

The salt in crystalline form has been found to be stable, highly soluble in water and easy to handle or process.

It has been found that 1-(indole-6-carbonyl-D-phenyl-glycinyl)-4-(1-methylpiperidin-4-yl)piperazine D-tartrate can be crystallised from various aqueous-organic solvent systems, including water/acetone and water/(1-4C)alkanol systems such as water/ethanol, water/n-propanol and water/iso-propanol. Particularly good yields have been obtained using water/acetone, especially in a volume ratio of about 15/85 water/acetone. Conveniently, 1-(indole-6-carbonyl-D-phenyl-glycinyl)-4-(1-methylpiperidin-4-yl)piperazine and one molar equivalent of D-tartaric acid are dissolved in water, then an organic solvent is added. Seeding with a small quantity of previously prepared crystals may help initiate crystallisation.

1-(Indole-6-carbonyl-D-phenylglycinyl)-4-(1-methyl-piperidin-4-yl)piperazine D-tartrate has been found to crystallise as thin plates, which are highly agglomerated and form a flowable powder of improved density.

The thermal stability and salvation state of the crystalline tartrate salt were determined by differential thermal/thermogravimetric analyses using a TA simultaneous TG/DTA unit. Samples were heated in open aluminum pans from 25 to ≧300° C. at 10° C./min with a nitrogen purge of 150 mL/min. The temperature was calibrated with indium. The weight calibration was performed with manufacturer-supplied standards and verified against sodium tartrate desolvation. The D-tartrate crystals were found to contain about 5-6% by weight of a solvent (predominantly water), which is consistent with the crystals being a dihydrate. As the dihydrate was heated above about 50° C., the water was lost. At about 145° C., the residual anhydrous solid melted. Upon recooling, the melt formed into an amorphous solid.

A moisture sorption isotherm of the D-tartrate crystals was also determined using a vacuum microbalance, with a 40° C. drying step prior to initial data collection. With the initial drying step, an initial 6% weight loss was observed, consistent with the removal of the waters of crystallization. As the relative humidity was increased, the sample resorbed water, with rehydration being completed when the relative humidity reached about 20%. Once the dihydrate had been formed, it remained stable between 5 and 95% relative humidity at ambient temperature.

Stability at low and high relative humidity is desirable in a product to be used or sold in a wide diversity of environments.

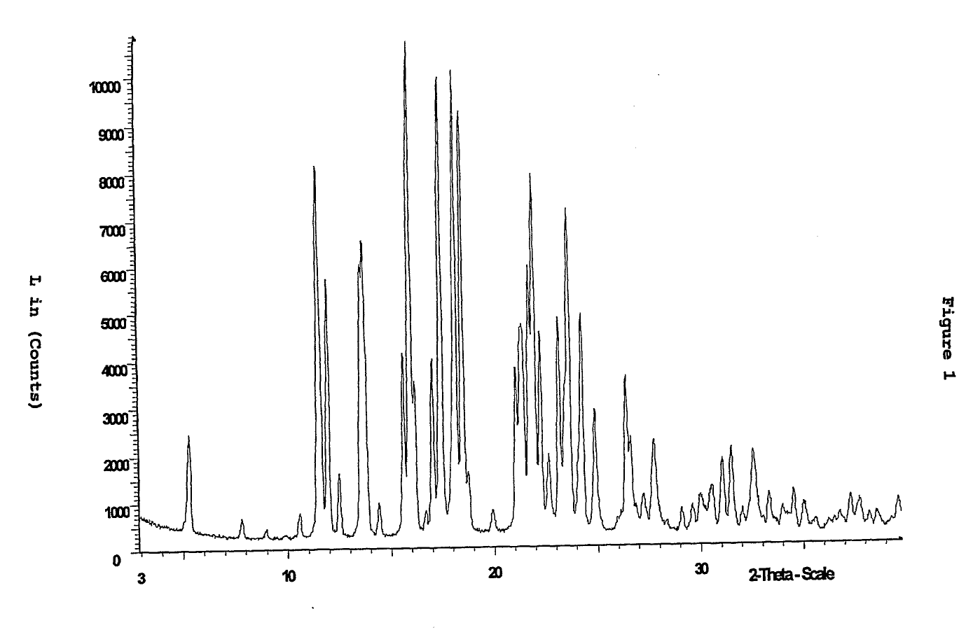

The crystalline material was subjected to X-ray powder diffraction analysis. The resultant X-ray powder diffraction pattern was found to contain sharp, intense peaks at ° 2θ=11.5, 15.9, 17.4, 18.1, 18.5 and 21.9. Peaks at ° 2θ=5.2 and 12.0 are also characteristic of this crystalline salt form. A more detailed analysis of the peaks is provided in Table 1 below. The X-ray powder diffraction pattern is shown in FIG. 1.

TABLE 1

| Angle (° 2θ) | I/Iₒ (%) |
|---|---|
| 5.2 | 22.8 |
| 11.5 | 75.8 |
| 12.0 | 53.4 |
| 12.5 | 14.8 |
| 13.7 | 61.1 |
| 15.6 | 38.3 |
| 15.9 | 100.0 |
| 16.2 | 32.8 |
| 17.0 | 37.1 |
| 17.4 | 92.7 |
| 18.1 | 94.1 |
| 18.5 | 86.2 |
| 18.8 | 14.8 |
| 21.1 | 35.2 |
| 21.4 | 43.3 |
| 21.7 | 55.7 |
| 21.9 | 73.7 |
| 22.2 | 42.2 |
| 22.6 | 18.2 |
| 23.1 | 45.2 |
| 23.6 | 66.7 |
| 24.3 | 45.8 |
| 24.9 | 26.8 |
| 26.4 | 33.2 |
| 26.6 | 21.3 |
| 27.7 | 20.7 |
| 30.6 | 11.5 |
| 31.1 | 16.8 |
| 31.5 | 18.9 |
| 32.6 | 18.4 |
| 34.6 | 10.6 |

The X-ray powder diffraction (XRD) pattern was obtained on a Siemens D5000 X-ray powder diffractometer, equipped with a CuKα source (λ=1.54056 Å) and a Kevex solid state Si(Li) detector, operating at 50 kV and 40 mA. The sample was scanned between 30° and 40° in 2θ, with a step size of 0.02° in 2θ and a scan rate of 9.0 seconds/step, and with 1 mm divergence and receiving slits and a 0.1 mm detector slit. The dry powder was packed onto a low background sample holder and a smooth surface was obtained using a glass slide.

It will be appreciated by those skilled in the art of X-ray powder diffraction analysis that the exact values measured for ° 2θ (or the corresponding d-spacings) may vary depending upon the particular sample analysed and the apparatus and particular analysis procedure used. An error of ±0.1° 2θ would be typical.

The D-tartrate salt crystals were also analyzed by solid-state ¹³C nuclear magnetic resonance (NMR) spectroscopy. Solid-state ¹³C chemical shifts reflect the molecular structure and electronic environment of the molecule in the crystal. The spectrum for the crystals is comprised of isotropic peaks for the drug cation and tartrate anion at the following chemical shifts: 25.3, 26.2, 42.6, 45.8, 46.2, 47.1, 48.3, 50.3, 52.1, 54.0, 55.5, 57.1, 61.8, 73.4, 74.2, 76.4, 101.5, 102.2, 112.9, 114.9, 117.3, 118.2, 119.6, 121.1, 125.6, 126.2, 127.4, 128.5, 129.5, 130.6, 132.2, 136.7, 139.5, 167.6, 169.0, 170.4, 174.9, 175.8, 178.6, and 179.2 ppm.

¹³C Cross polarization/magic angle spinning (CP/MAS) NMR (solid-state NMR or SSNMR) spectra were obtained using a Varian Unity Inova 400 MHz NMR spectrometer operating at a carbon frequency of 100.573 MHz and equipped with a complete solids accessory and a Chemagnetics 4.0 mm T3 probe. Ramped-amplitude cross polarization (RAMP-CP) at 62 kHz and TPPM decoupling at 70 kHz were used. Acquisition parameters were as follows: 90° proton r.f. pulse width 4.0 μs, contact time 3.0 ms, pulse repetition time 10 s, MAS frequency 10.0 kHz, spectral width 50 kHz, and acquisition time 50 ms. Chemical shifts were referenced to the methyl group of hexamethylbenzene (δ=17.3 ppm) by sample replacement.

The equilibrium solubilities of the D-tartrate crystals in water and in 0.01 M HCl were measured at 25° C. and were found to be ≧126 mg/mL and ≧125 mg/mL respectively (measured as the free base).

Thus it has been found that the D-tartrate salt affords a crystalline material that is stable, has excellent processing properties and has high water solubility.

It will be understood that the D-tartrate salt according to the invention may be isolated in the form of a solvate, such as the dihydrate, and that all such solvates are therefore included within the scope of the present invention. It will be appreciated that a solvate that is not physiologically tolerable may nevertheless be useful in the manufacture of a pharmaceutical product, for example in a purification step.

The D-tartrate salt of the invention may be administered by any convenient route, e.g. into the gastrointestinal tract (e.g. rectally or orally), the nose, lungs, musculature or vasculature or transdermally. The D-tartrate salt may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. Preferably, for injection or infusion, the compositions will be sterile and in a suitable solution or suspension form. Such compositions form a further aspect of the invention.

Viewed from this aspect the invention provides a pharmaceutical composition comprising the D-tartrate salt according to the invention together with at least one pharmaceutically acceptable carrier or excipient. The pharmaceutical composition may also optionally comprise at least one further antithrombotic and/or thrombolytic agent. The compound may, with benefit, form part of a combination therapy with an anticoagulant with a different mode of action or with a thrombolytic agent.

Viewed from a further aspect the invention provides the use of the D-tartrate salt according to the invention for the manufacture of a medicament for use in a method of treatment of the human or non-human animal body (e.g. a mammalian body in a sensitive species) to combat (i.e. treat or prevent) a condition responsive to said inhibitor (e.g. a thrombotic disorder as described hereinabove).

Viewed from a further aspect the invention provides a method of treatment of the human or non-human animal body (e.g. a mammalian body in a sensitive species) to combat a condition responsive to a Factor Xa inhibitor (e.g. a thrombotic disorder as described hereinabove), said method comprising administering to said body an effective amount of the D-tartrate salt according to the invention.

The dosage of the compound of the invention will depend upon the nature and severity of the condition being treated, the administration route and the size and species of the patient. However in general, quantities of from 0.01 to 100 μmol/kg bodyweight will be administered.

All publications referred to herein are hereby incorporated by reference.

The invention will now be described further with reference to the following non-limiting Examples.

### EXPERIMENTAL

1-(Indole-6-carbonyl-D-phenylglycinyl)-4-(1-methyl-piperidin-4-yl)piperazine may be obtained, for example, following the method described in WO 01/96323.

### Example 1

### Preparation of 1-(Indole-6-carbonyl-D-phenylglycinyl)-4-(1-methylpiperidin-4-yl)piperazine D-Tartrate Dihydrate

1-(Indole-6-carbonyl-D-phenylglycinyl)-4-(1-methyl-piperidin-4-yl)piperazine (50 mg) and D-tartaric acid (0.5 molar equivalent, about 8.3 mg) are dissolved in anhydrous ethanol (1 mL) with mild heating. Upon cooling, some fine solid/cloudiness is observed. A few drops of water are added, and the clear solution is stirred at room temperature. After 4 days, the precipitate which has formed is isolated by vacuum filtration to provide the named product (typically, 30 mg) as a white powder.

Alternatively, 1-(indole-6-carbonyl-D-phenylglycinyl)-4-(1-methylpiperidin-4-yl)piperazine (200 mg) and D-tartaric acid (one molar equivalent, 65 mg) are dissolved in water (2 mL) at room temperature. Some haziness in the solution is removed by gravity filtration. Isopropanol (8 mL) is added to the clear solution until haziness persists. After about 15 to 20 min, some sticky solid is observed, with subsequent formation of a white slurry. After the slurry has thickened, additional isopropanol (2 mL) is added; and the slurry is maintained at room temperature overnight. The solid precipitate is isolated by vacuum filtration and washed with isopropanol before it is dried in a convection oven at 70° C. for 1 h to afford the title product (typical yield, 190 mg).

### Example 2

### Preparation of 1-(Indole-6-carbonyl-D-phenylglycinyl)-4-(1-methylpiperidin-4-yl)piperazine D-Tartrate Dihydrate

To 1-(indole-6-carbonyl-D-phenylglycinyl)-4-(1-methyl-piperidin-4-yl)piperazine (46.0 g, 0.1 mol) and D-tartaric acid (15.0 g, 0.1 mol, Aldrich 99% ee) is added water (230 mL) and acetone (800 mL). The mixture is stirred at room temperature for 15 min during which the materials dissolve to afford a clear solution. To this solution, crystal seeds of 1-(indole-6-carbonyl-D-phenylglycinyl)-4-(1-methylpiperidin-4-yl)piperazine D-tartrate dihydrate (100 mg) are added in one portion. To this slurry, a second part of anti-solvent acetone (800 mL) is added at a very slow rate (60-100 mL/hour). After the addition is complete, the suspension is stirred for 4 h at room temperature and then cooled in an ice-water bath to 5° C. for another 2 h.

Crystals are collected and washed with cold acetone (100 mL). The crystals are dried under vacuum (about 15 mm, 2 kPa) at 40° C. for 24 h to provide the title salt (55.7 g, 86.8%). Alternatively, the crystals may be blown dry with nitrogen to avoid removing the waters of hydration in the product.

1. 1-(Indole-6-carbonyl-D-phenylglycinyl)-4-(1-methylpiperidin-4-yl)piperazine D-tartrate.

2. 1-(Indole-6-carbonyl-D-phenylglycinyl)-4-(1-methylpiperidin-4-yl)piperazine D-tartrate in crystalline form.

3. 1-(Indole-6-carbonyl-D-phenylglycinyl)-4-(1-methylpiperidin-4-yl)piperazine D-tartrate in crystalline form having an X-ray powder diffraction pattern with sharp, intense peaks at ° 2θ=11.5, 15.9, 17.4, 18.1, 18.5 and 21.9.

4. (canceled)

5. (canceled)

6. (canceled)

7. A method of treatment of a human or non-human animal body to combat a thrombotic disorder, which comprises administering to said body an effective amount of the D-tartrate salt as claimed in claim 1.

8. A method of treatment of a human or non-human animal body to combat a thrombotic disorder, which comprises administering to said body an effective amount of the D-tartrate salt as claimed in claim 2.

9. A method of treatment of a human or non-human animal body to combat a thrombotic disorder, which comprises administering to said body an effective amount of the D-tartrate salt as claimed in claim 3.

10. (canceled)

11. A pharmaceutical composition, which comprises the D-tartrate salt as claimed in claim 1 together with at least one pharmaceutically acceptable carrier or excipient.

12. A pharmaceutical composition, which comprises the D-tartrate salt as claimed in claim 2 together with at least one pharmaceutically acceptable carrier or excipient.

13. A pharmaceutical composition, which comprises the D-tartrate salt as claimed in claim 3 together with at least one pharmaceutically acceptable carrier or excipient.
